(19) United States
(12) Patent Application Publication
(10) Pub. No.: US 2009/0198212 A1
(43) Pub. Date: Aug. 6, 2009

(54) Endoscopic injection needle assembly inluding an endoscopic hood

(76) Inventors: Tyler Timberlake, Boyce, VA (US); William Lucas Churchill, Bolton, MA (US); Stephen J. Perry, Shirley, MA (US)

Correspondence Address: KRIEGSMAN & KRIEGSMAN, 30 TURNPIKE ROAD, SUITE 9, SOUTHBOROUGH, MA, 01772, US

(21) Appl. No.: 12/152,833

(22) Filed: May 16, 2008

Related U.S. Application Data
(60) Provisional application No. 60/930,384, filed on May 16, 2007.

(51) Int. Cl. A61B 1/018; A61M 5/00

(52) U.S. Cl. 604/506; 600/104

(57) ABSTRACT

An endoscopic injection needle assembly and method of using the same. According to one embodiment, the assembly includes an endoscope, an injection needle, an endoscopic hood and a suction source. The endoscope is shaped to include a proximal end, a distal end, and a plurality of longitudinal channels. The distal end of the injection needle is positioned within a working channel of the endoscope. The injection needle is sized relative to the working channel to permit fluid to flow around the injection needle in the working channel. The endoscopic hood is mounted on the distal end of the endoscope and extends distally therefrom, the endoscopic hood being in fluid communication with the working channel and having an opening through which fluid may be drawn into the endoscopic hood. The suction source is applied to the proximal end of the working channel to cause fluid to be drawn into the endoscopic hood through the opening and then proximally through the working channel. In addition, suction causes tissue to be drawn into the endoscopic hood. The injection needle may then be operated to penetrate the suctioned tissued.

### CROSS-REFERENCE TO RELATED APPLICATIONS

The present application claims the benefit under 35 U.S.C. 119(e) of U.S. Provisional Patent Application Ser. No. 60/930,384, filed May 16, 2007, the disclosure of which is incorporated herein by reference.

### BACKGROUND OF THE INVENTION

The present invention relates generally to endoscopic injection needle assemblies.

Nearly half of all Americans suffer from heartburn at least once a month. Heartburn occurs when stomach fluids and acids escape from the stomach and enter into the esophagus, irritating the esophagus. Normally, a muscular ring called the lower esophageal sphincter (LES) acts as a valve between the esophagus and the stomach to allow food to pass from the esophagus into the stomach while keeping stomach fluids and acids from escaping from the stomach into the esophagus. In those instances in which the LES fails to keep stomach fluids and acids in the stomach, heartburn occurs.

For some people who suffer from heartburn, the heartburn is severe enough or frequent enough to disrupt their daily activities and/or their sleep. Such a condition is called gastroesophageal reflux disease (GERD). In some people who have GERD, the LES relaxes more than it should and/or at the wrong times and causes acid to pass into the esophagus, which causes chronic heartburn.

In addition to causing frequent and/or severe heartburn, GERD can cause other health problems. For example, the fluids and acids that reflux into the esophagus can lead to inflammation of the esophagus (esophagitis) or ulcers. In severe cases, this damage can scar the esophageal lining and narrow it, causing a stricture which may make it hard or painful for the patient to swallow. In certain cases, this may lead to a condition called Barrett's esophagus, where the lining of the esophagus changes and may over time lead to cancer of the esophagus.

Many people can get relief from GERD symptoms by changing their diet and/or using appropriate medications. Some of the medications available for managing GERD symptoms include common antacids as well as drugs that slow down the production of stomach acids, such as proton pump inhibitors and H₂ receptor antagonists.

However, it should be noted that medications of the type described above merely address symptoms of GERD and do not address the condition's mechanical etiology. Thus, GERD symptoms often recur after drug withdrawal. In addition, while medications may effectively treat the acid-induced symptoms of GERD, they do not treat alkaline reflux, which may also result in esophageal mucosal injury.

Since GERD is a chronic condition, it may be necessary for a patient to take medications for the rest of his/her life to obtain relief from GERD symptoms. However, for many patients, the expense and the psychological burden of a lifetime of medication dependence, as well as the uncertainty of long-term effects of some newer medications, make surgical treatment an alluring alternative to a medicinal approach. However, surgical intervention, often in the form of anti-reflux surgery, is a major undertaking and includes its own set of risks.

Fortunately, a minimally invasive technique has been devised for treating GERD. This technique, which is more fully disclosed in U.S. Pat. Nos. 6,238,335, 6,251,063, 6,251,064 and 6,695,764, all of which are incorporated herein by reference, typically involves first inserting an endoscope down through the patient's mouth and into the esophagus in proximity to the LES. Then, the distal end of a device commonly referred to as “an injection needle” is inserted through a working channel of the endoscope until a needle at the distal end of the injection needle is inserted into the muscle of the LES. Then, a special solution is dispensed through the injection needle and into the muscle of the LES. The solution, which is commercially available from Boston Scientific Corporation (Natick, Mass.) as Enteryx® solution, includes a biocompatible polymer that forms a soft, spongy, permanent implant in the sphincter muscle that helps the LES to keep stomach fluids and acids from backing up into the esophagus.

Typically, an injection needle of the type referred to above comprises a hollow needle, a flexible inner catheter, a flexible outer catheter, an inner hub and an outer hub. The proximal end of the hollow needle is typically fixedly mounted within the distal end of the flexible inner catheter. The inner hub is typically fixedly mounted on the proximal end of the inner catheter and is adapted to convey fluids to the inner catheter from a needleless syringe or the like. The inner catheter and the hollow needle are typically slidably mounted within the outer catheter so that one may extend the hollow needle out of the distal end of the outer catheter when one wishes to make an injection and retract the hollow needle into the outer catheter when not making an injection. The outer hub is typically fixedly mounted on the proximal end of the outer catheter and is adapted to engage the inner hub so as to limit the distal movement of the needle and the inner catheter relative to the outer catheter. Examples of injection needles are disclosed in the following patents, all of which are incorporated herein by reference: U.S. Pat. No. 6,770,053; U.S. Pat. No. 6,585,694; U.S. Pat. No. 6,423,034; U.S. Pat. No. 6,401,718; U.S. Pat. No. 6,336,915; U.S. Pat. No. 5,785,689; U.S. Pat. No. 4,946,442; and U.S. Pat. No. 4,668,226.

### SUMMARY OF THE INVENTION

According to one aspect of the invention, there is provided a novel endoscopic injection needle assembly, said endoscopic injection needle assembly comprising (a) an endoscope, said endoscope including a working channel, said working channel having a proximal end and a distal end; (b) an injection needle, said injection needle being disposed within said working channel, said injection needle being sized relative to said working channel to permit fluid to flow around said injection needle in said working channel; (c) a hood, said hood having a proximal end and a distal end, said hood extending distally from said distal end of said endoscope, said hood being in fluid communication with said working channel and having an opening through which fluid may be drawn into said hood; and (d) a suction source, said suction source being applied to said proximal end of said working channel to cause fluid to be drawn into said hood through said opening and then proximally through said working channel.

According to another aspect of the invention, there is provided a method of making an injection, said method comprising the steps of (a) providing an endoscope assembly, the endoscope assembly including an endoscope and a hood, the endoscope including a proximal end, a distal end and a working channel extending from the proximal end of the endoscope to the distal end of the endoscope, the hood being mounted on the endoscope and extending distally beyond the distal end of the endoscope, the hood having a proximal end, a distal end, and an opening through which fluid may be drawn into the hood; (b) inserting the hood and the distal end of the endoscope into a patient; (c) positioning the hood in the area of a targeted tissue; (d) providing an injection needle, the injection needle having a proximal end and a distal end and including a hollow needle and a catheter, the catheter being fluidly coupled to the hollow needle, the distal end of the injection needle being positioned within the working channel of the endoscope so that the hollow needle is in the vicinity of the distal end of the endoscope, the injection needle being sized relative to the working channel to permit fluid to flow around the injection needle in the working channel; (e) applying a vacuum to a proximal end of the working channel to draw fluid into the hood through the opening and then proximally through the working channel of the endoscope, thereby causing tissue to be drawn into the hood; (f) inserting the hollow needle of the injection needle into the tissue drawn into the hood; and (g) dispensing a desired substance into the inserted tissue through the catheter and the hollow needle.

For purposes of the present specification and claims, various relational terms like “top,” “bottom,” “proximal,” “distal,” “upper,” “lower,” “front,” and “rear” are used to describe the present invention when said invention is positioned in or viewed from a given orientation. It is to be understood that, by altering the orientation of the invention, certain relational terms may need to be adjusted accordingly.

Various objects, features and advantages of the present invention will be set forth in part in the description which follows, and in part will be obvious from the description or may be learned by practice of the invention. In the description, reference is made to the accompanying drawings which form a part thereof and in which is shown by way of illustration various embodiments for practicing the invention.

### BRIEF DESCRIPTION OF THE DRAWINGS

The accompanying drawings, which are hereby incorporated into and constitute a part of this specification, illustrate various embodiments of the invention and, together with the description, serve to explain the principles of the invention. In the drawings wherein like reference numerals represent like parts:

FIGS. 1(a) through 1(c) are fragmentary schematic side, fragmentary perspective, and enlarged fragmentary longitudinal section views, respectively, of a first embodiment of an endoscopic injection needle assembly constructed according to the teachings of the present invention;

FIGS. 2(a) and 2(b) are fragmentary schematic side and fragmentary longitudinal section views, respectively, showing how the endoscopic injection needle assembly of FIGS. 1(a) through 1(c) may be used;

FIG. 3 is a fragmentary side view of a second embodiment of an endoscopic injection needle assembly constructed according to the teachings of the present invention;

FIG. 4 is a fragmentary longitudinal section view, showing how the endoscopic injection needle assembly of FIG. 3 may be used;

FIG. 5 is a fragmentary side view of a third embodiment of an endoscopic injection needle assembly constructed according to the teachings of the present invention;

FIG. 6 is a fragmentary longitudinal section view, showing how the endoscopic injection needle assembly of FIG. 5 may be used;

FIGS. 7(a) and 7(b) are distal end and fragmentary longitudinal section views, respectively, of a fourth embodiment of an endoscopic injection needle assembly constructed according to the teachings of the present invention;

FIG. 8(a) is a fragmentary perspective view of a fifth embodiment of an endoscopic injection needle assembly constructed according to the teachings of the present invention;

FIG. 8(b) is a longitudinal view, partly in section, of the endoscopic injection needle assembly of FIG. 8(a);

FIG. 9 is a longitudinal view, partly in section, showing how the endoscopic injection needle assembly of FIGS. 8(a) and 8(b) may be used;

FIG. 10 is a fragmentary perspective view of a sixth embodiment of an endoscopic injection needle assembly constructed according to the teachings of the present invention;

FIG. 11 is a fragmentary perspective view of a seventh embodiment of an endoscopic injection needle assembly constructed according to the teachings of the present invention;

FIG. 12 is a fragmentary exploded perspective view of an eighth embodiment of an endoscopic injection needle assembly constructed according to the teachings of the present invention;

FIG. 13 is a fragmentary perspective view of a ninth embodiment of an endoscopic injection needle assembly constructed according to the teachings of the present invention; and

FIG. 14 is a fragmentary longitudinal section view of a tenth embodiment of an endoscopic injection needle assembly constructed according to the teachings of the present invention.

### DETAILED DESCRIPTION OF PREFERRED EMBODIMENTS

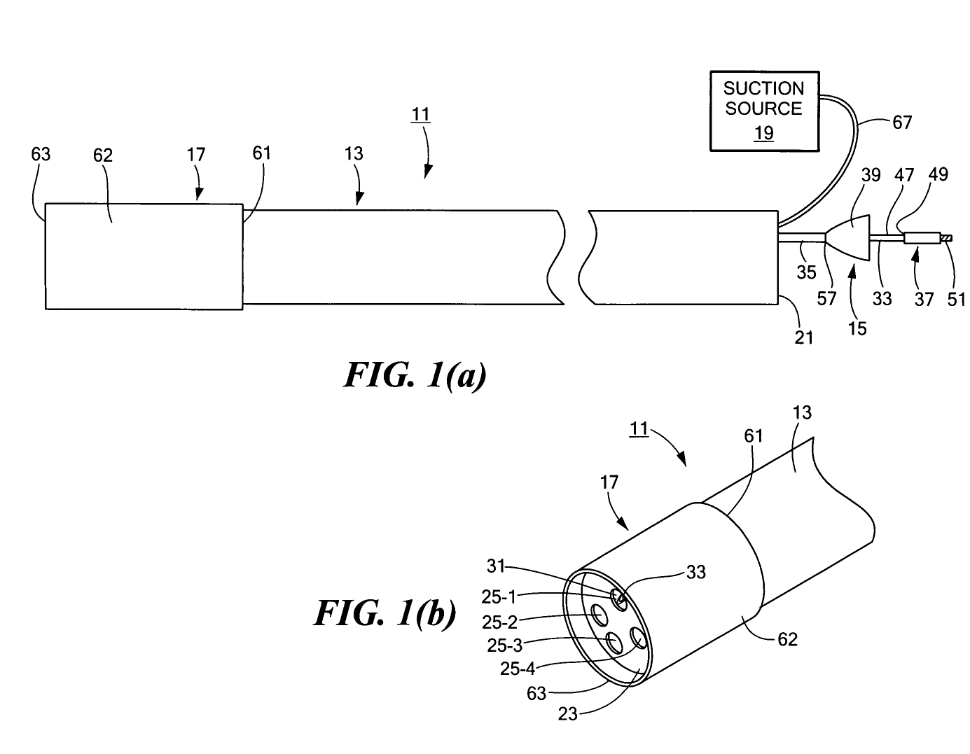

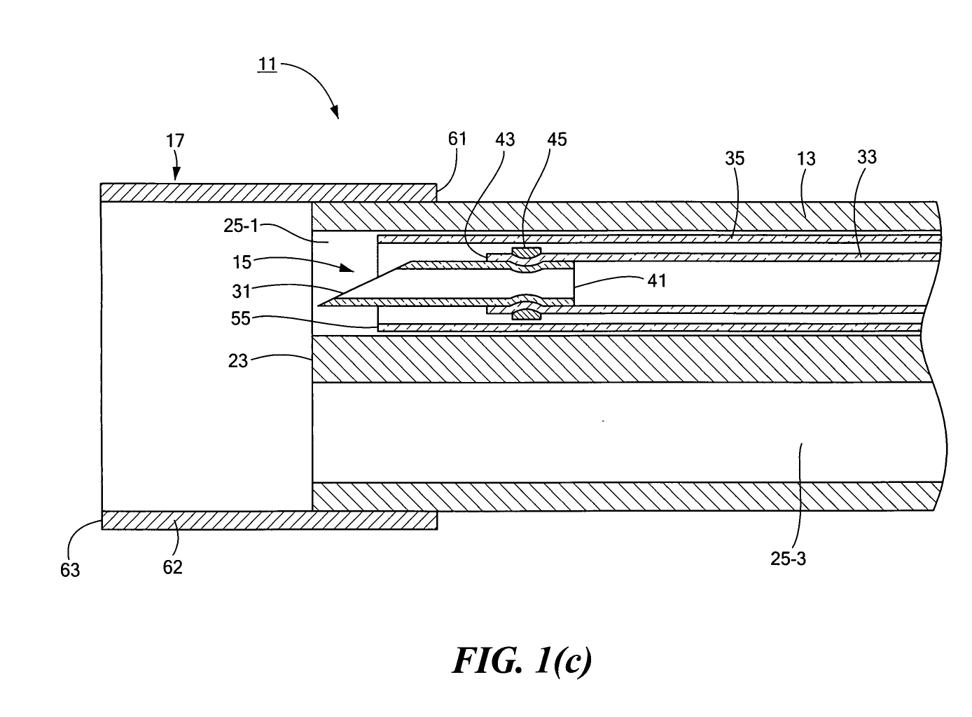

Referring now to FIGS. 1(a) through 1(c), there are shown fragmentary schematic side, fragmentary perspective, and enlarged fragmentary longitudinal section views, respectively, of a first embodiment of an endoscopic injection needle assembly constructed according to the teachings of the present invention, said endoscopic injection needle assembly being represented generally by reference numeral 11.

Assembly 11 may comprise an endoscope 13, an injection needle 15, an endoscopic hood 17, and a suction source 19.

Endoscope 13, which may be a conventional endoscope, may be an elongated, flexible member having a proximal end 21 and a distal end 23. Endoscope 13 may be shaped to include a plurality of longitudinal channels 25-1 through 25-4. For example, channel 25-1 may be used as a working channel, channel 25-2 may be used as a water or irrigation channel, channel 25-3 may be used as an optical or viewing channel, and channel 25-4 may be used as a fluid channel to blow air or another suitable fluid onto a target.

Injection needle 15, which may be a conventional injection needle, may comprise a hollow needle 31, a flexible inner catheter 33, a flexible outer catheter 35, a tubular inner hub 37 and a tubular outer hub 39. The proximal end 41 of hollow needle 31 may be fixedly mounted within the distal end 43 of flexible inner catheter 33 by a metal band 45 that may be crimped around the outside of inner catheter 33. The proximal end 47 of inner catheter 33 may be fixedly mounted within the distal end 49 of inner hub 37. The proximal end 51 of inner hub 37 may be externally threaded and may be adapted for connection to a conventional needleless syringe or the like. Inner catheter 33 and hollow needle 31 may be slidably mounted within outer catheter 35 so that one may extend hollow needle 31 out of the distal end 55 of outer catheter 35 when one wishes to make an injection and may retract hollow needle 31 into outer catheter 35 when not making an injection. Outer hub 39 may be fixedly mounted over the proximal end 57 of outer catheter 35 and may be adapted to engage inner hub 37 so as to limit the distal movement of needle 31 and inner catheter 33 relative to outer catheter 35.

Injection needle 15 may be removably mounted in working channel 25-1 of endoscope 13, with the distal end of injection needle 15 (e.g., needle 31, distal end 43 of inner catheter 33, distal end 55 of outer catheter 35) being positioned in the vicinity of distal end 23 of endoscope 13 and with inner hub 37 and outer hub 39 preferably not being inserted into working channel 25-1. For reasons to become apparent below, outer catheter 35 may be appropriately dimensioned relative to working channel 25-1 so that, with injection needle 15 loaded into channel 25-1, fluid may be drawn proximally through channel 25-1 around the outside of outer catheter 35. For example, as is shown in FIG. 1(c), outer catheter 35 may be undersized relative to working channel 25-1. Alternatively, injection needle 15 and/or working channel 25-1 may be designed to allow fluid flow around injection needle 15, for example, by having small grooves or channels or by having a non-round or other appropriate shape.

Endoscopic hood 17 may be a flexible, unitary, tubular member having an open proximal end 61, a cylindrical wall 62, and an open and straight distal end 63. (Instead of being flexible, hood 17 may be rigid along its entire length or may be rigid over some of its length with a flexible seal at its distal end.) Proximal end 61 of hood 17 may be fixedly mounted, such as by a friction-fit, over distal end 23 of endoscope 13, with distal end 63 of hood 17 extending distally a short distance beyond distal end 23 of endoscope 13. (Alternatively, instead of being mounted by a friction-fit over distal end 23 of endoscope 13, hood 17 may be secured to endoscope 13 by a snap-fit, screw threads, slots or notches in hood 17 to allow it to fit around endoscope 13, more flexible material on proximal end of hood 17, O-rings, two halves snapping around endoscope 13, etc.) Distal end 63 of hood 17 may be designed to contact tissue in such a way as to optimize suction and, accordingly, may be flat or may have rounded edges. In addition, to improve sealing between distal end 63 of hood 17 and tissue, wall 62 may be provided with one or more longitudinal lumina through which suction may be applied.

Suction source 19, which may be a conventional vacuum or the like, may be coupled to a length of tubing 67, the distal end of tubing 67 being inserted into the proximal end of channel 25-1 alongside of injection needle 15. In this manner, suction source 19 may be used to draw fluid proximally into and through endoscopic hood 17 and channel 25-1.

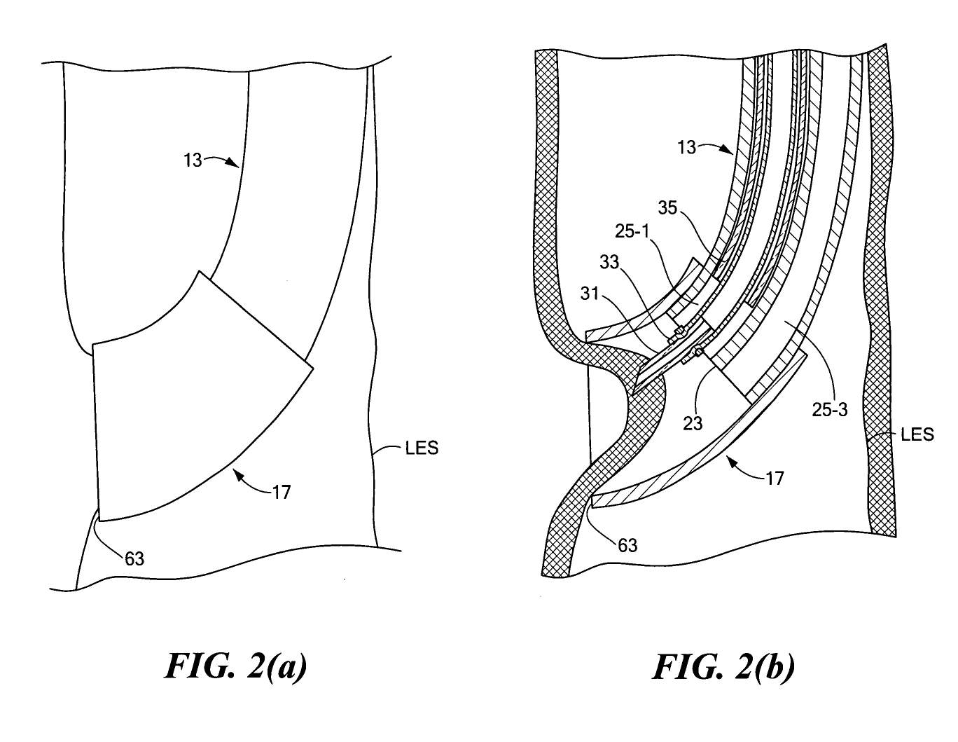

To use assembly 11, for example, to inject implant material into the lower esophageal sphincter of a patient, one first may insert distal end 23 of endoscope 13 through the mouth of the patient and then into the esophagus of the patient in the vicinity of the lower esophageal sphincter (LES). One then may load injection needle 15 into working channel 25-1 of endoscope 13, with the distal end of injection needle 15 being positioned in the vicinity of distal end 23 of endoscope 13 and with needle 31 retracted into outer catheter 35. Next, endoscope 13 may be manipulated so that distal end 63 of endoscopic hood 17 is pressed firmly against the LES tissue surrounding the targeted injection site. If desired, a lubricant may be applied to distal end 63 of hood 17 to promote sealing between hood 17 and the tissue pressed thereagainst. Next, tubing 67 may be inserted into the proximal end of channel 25-1, and suction source 19 may be turned on, causing the tissue circumscribed by hood 17 to be sucked proximally into hood 17 (see FIGS. 2(a) and 2(b)). (Instead of using suction source 19 and tubing 67 to create suction, one may use a syringe.) With the tissue thus drawn into hood 17, needle 31 may be extended distally from outer catheter 35 and into hood 17, where it may pierce the tissue that has been sucked into hood 17. The implant material may then be injected into the penetrated tissue through needle 31 in the conventional manner. After the injection is complete, needle 31 may be withdrawn, and the suctioned tissue may be released.

One particularly desirable feature about assembly 11 is that the use of suction, coupled with the pressing of distal end 63 of endoscopic hood 17 against the tissue surrounding the targeted insertion site, may serve to stabilize such tissue so that injection needle 15 may more easily be inserted thereinto. In the absence of such stabilization, the injection needle tends (i) to push the tissue away without penetrating the tissue; (ii) to lose penetration of the tissue; and/or (iii) to pass entirely through the tissue.

Another particularly desirable feature about assembly 11 is that one can control the depth of penetration of needle 31 into the targeted tissue by adjusting the distance that distal end 63 of endoscopic hood 17 extends beyond distal end 23 of endoscope 13. In other words, generally speaking, the greater the distance that endoscopic hood 17 extends beyond distal end 23 of endoscope 13, the greater the depth of tissue penetration.

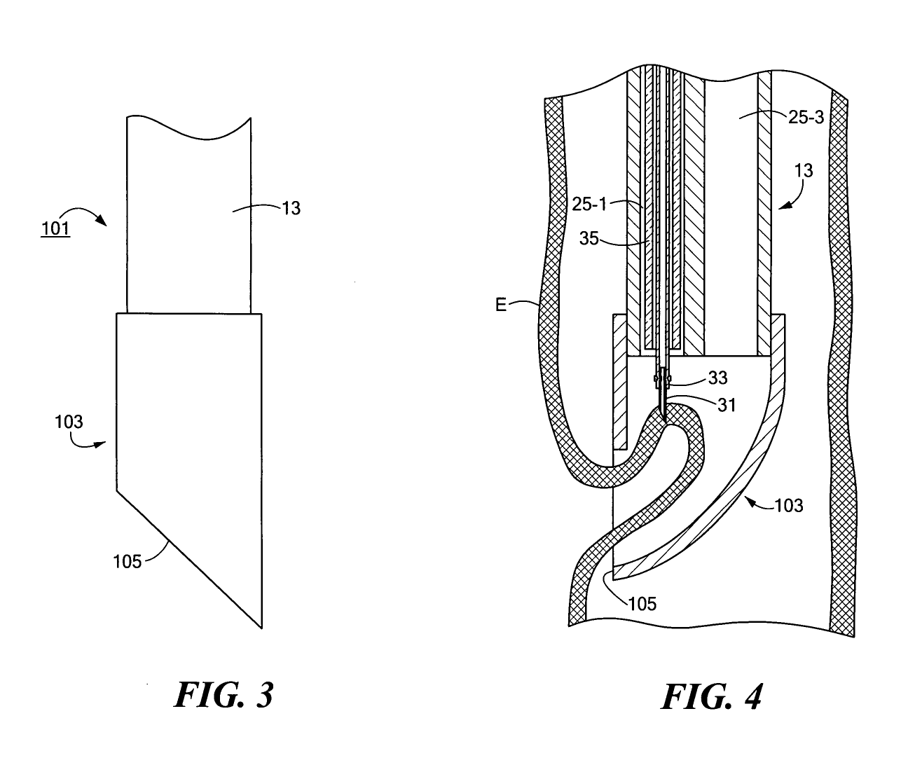

Referring now to FIG. 3, there is shown a fragmentary side view of a second embodiment of an endoscopic injection needle assembly constructed according to the teachings of the present invention, said endoscopic injection needle assembly being represented generally by reference numeral 101.

Assembly 101 may be similar in many respects to assembly 11, the principal difference between the two assemblies being that, whereas assembly 11 may include endoscopic hood 17, assembly 101 may include an endoscopic hood 103. Endoscopic hood 103 differs primarily from endoscopic hood 17 in that endoscopic hood 103 may have a beveled distal end 105 whereas endoscopic hood 17 may have a straight distal end 63. Because of its beveled shape, distal end 105 of hood 103 may be more easily pressed flush against the esophagus E (see FIG. 4) or other tissue, without requiring as much bending of endoscope 13 as may be required for straight distal end 63 of hood 17.

Assembly 101 may be used in a fashion similar to that discussed above in connection with assembly 11. Like assembly 11, assembly 101 may provide one with the ability to stabilize the targeted tissue and to control the depth of needle penetration.

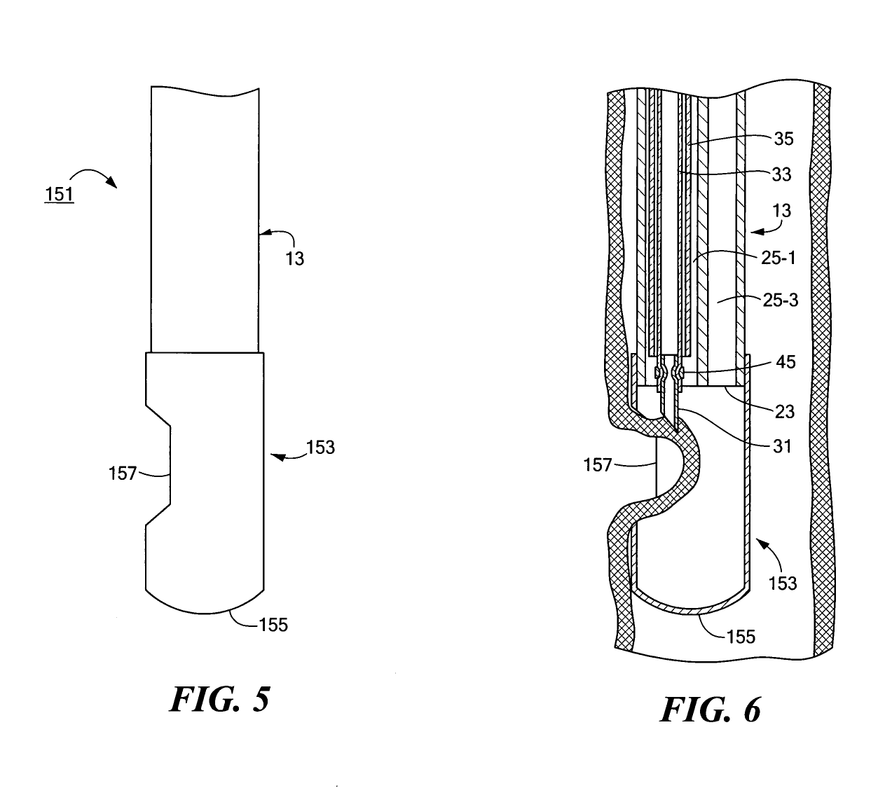

Referring now to FIG. 5, there is shown a fragmentary side view of a third embodiment of an endoscopic injection needle assembly constructed according to the teachings of the present invention, said endoscopic injection needle assembly being represented generally by reference numeral 151.

Assembly 151 may be similar in many respects to assembly 11, the principal difference between the two assemblies being that, whereas assembly 11 may include endoscopic hood 17, assembly 151 may include an endoscopic hood 153. Endoscopic hood 153 differs primarily from endoscopic hood 17 in that endoscopic hood 153 may have a closed and rounded distal end 155 and a side window 157 whereas endoscopic hood 17 may have an open and straight distal end 63 and may not have a side window. Assembly 151 may be used in a fashion similar to that discussed above in connection with assembly 11, except that hood 153 may be oriented relative to the esophagus so that side window 157 may be placed flush against the esophageal or other tissue wall, thereby permitting tissue to be drawn therethrough. As can be appreciated, because of the placement of side window 157 on hood 153, tissue may be sucked into hood 153 with minimal bending of endoscope 13 (see FIG. 6). Like assembly 11, assembly 151 may provide one with the ability to stabilize the targeted tissue and to control the depth of needle penetration.

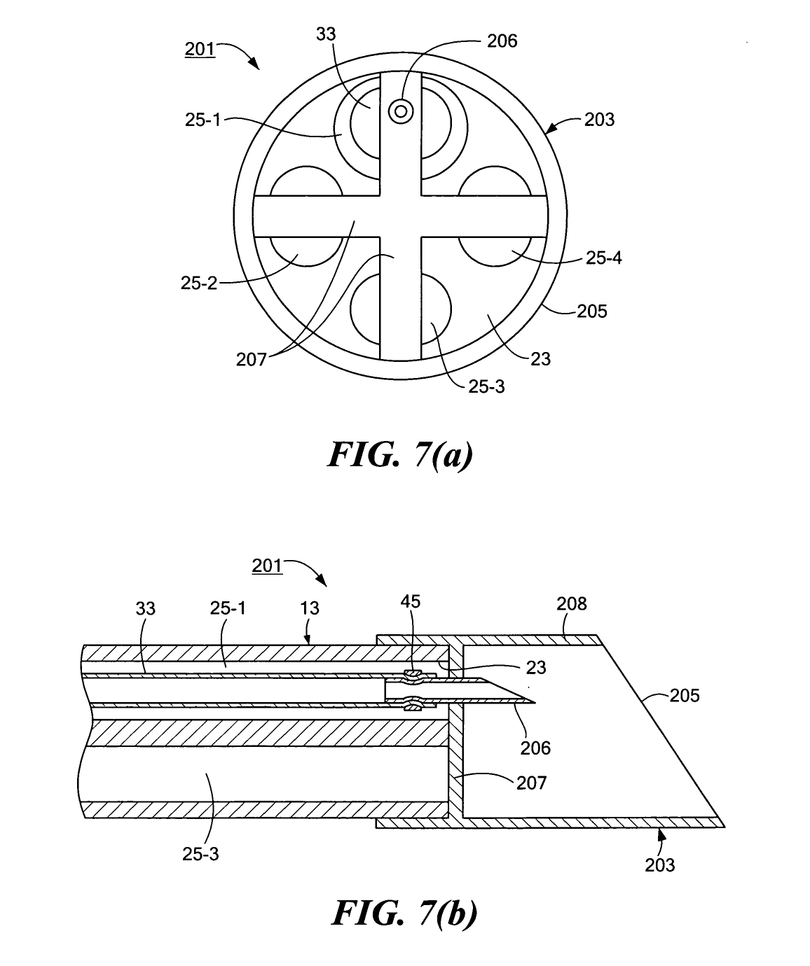

Referring now to FIGS. 7(a) and 7(b), there are shown distal end and fragmentary longitudinal section views, respectively, of a fourth embodiment of an endoscopic injection needle assembly constructed according to the teachings of the present invention, said endoscopic injection needle assembly represented generally by reference numeral 201.

Assembly 201 may be similar in certain respects to assembly 11, the principal differences between the two assemblies being that (i) whereas assembly 11 may include hood 17, which may have a straight distal end 63, assembly 201 may include hood 203, which, like hood 103, may have a beveled distal end 205; and (ii) whereas needle 31 of assembly 11 may be adapted for slidable movement relative to hood 17, assembly 201 may include a hollow needle 206 that may be fixedly mounted within endoscopic hood 203 by a plurality of slats 207 extending radially inwardly from a side wall 208. Accordingly, assembly 201 may not include an outer catheter corresponding to outer catheter 35.

Assembly 201 may be used in a fashion similar to that discussed above for assembly 11, except that, because of the fixed placement of needle 206 within hood 203, needle 206 may automatically pierce the tissue that may be drawn into hood 203, without needing to be moved into contact with the tissue. Like assembly 11, assembly 201 may provide one with the ability to stabilize the targeted tissue and to control the depth of needle penetration. The depth of injection may be controlled by the distance between the beveled edge 205 of hood 203 and needle 206. Slats 207 limit the volume of tissue drawn into hood 203.

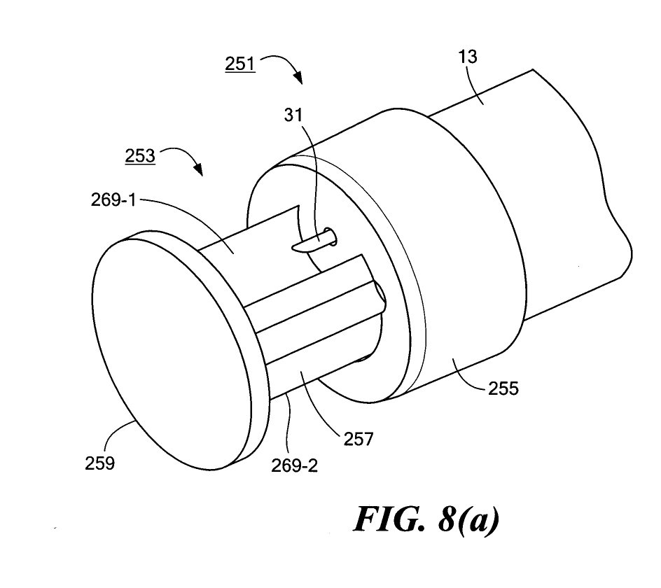

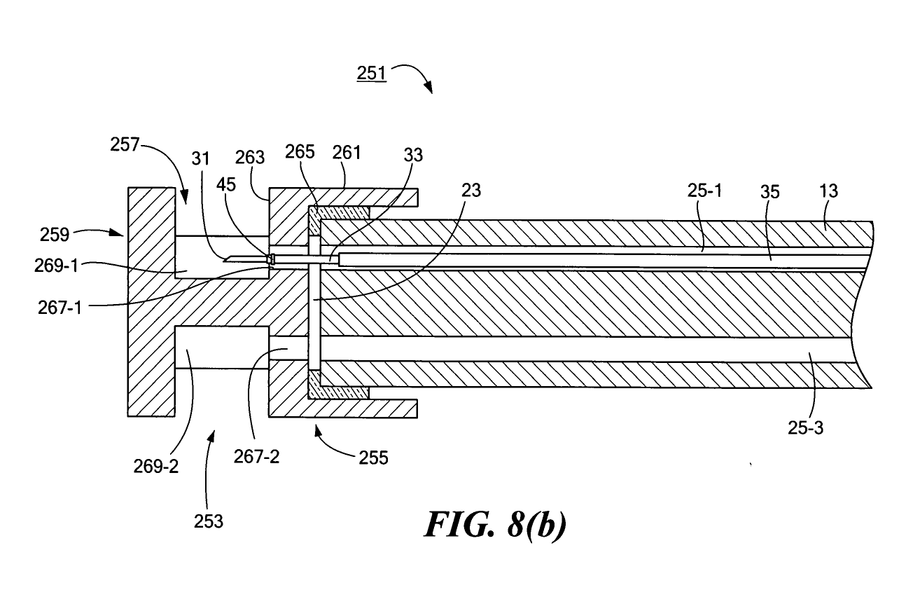

Referring now to FIGS. 8(a) and 8(b), there are shown fragmentary perspective and fragmentary longitudinal section views, respectively, of a fifth embodiment of an endoscopic injection needle assembly constructed according to the teachings of the present invention, said endoscopic injection needle assembly being represented generally by reference numeral 251.

Assembly 251 may be similar in many respects to assembly 11, the principal difference between the two assemblies being that, whereas assembly 11 may include endoscopic hood 17 fixedly mounted on distal end 23 of endoscope 13, assembly 251 may include an endoscopic hood 253 fixedly mounted on distal end 23 of endoscope 13. Hood 253 may be a unitary member shaped to include a proximal portion 255, an intermediate portion 257 and a distal portion 259. Proximal portion 255, which may be shaped to include a circular side wall 261 and a transverse wall 263, may be appropriately dimensioned to receive distal end 23 of endoscope 13. A gasket 265 may be positioned between endoscope 13 and proximal portion 255, gasket 265 serving both to securely mount proximal portion 255 on endoscope 13 and to space transverse wall 263 of proximal portion 255 from distal end 23 of endoscope 13. A pair of openings 267-1 and 267-2 may be provided in transverse wall 263. Opening 267-1 may be aligned with channel 25-1 and may be appropriately dimensioned for needle 31 to be inserted therethrough. Opening 267-2 may be aligned with channel 25-3, which may be a viewing channel.

Intermediate portion 257 of hood 253 may have a reduced outer diameter as compared to those of proximal portion 255 and distal portion 259. In addition, intermediate portion 257 may be shaped to include a pair of opposing concave surfaces 269-1 and 269-2, surface 269-1 being shaped to permit access to opening 267-1, surface 269-2 being shaped to permit access to opening 267-2.

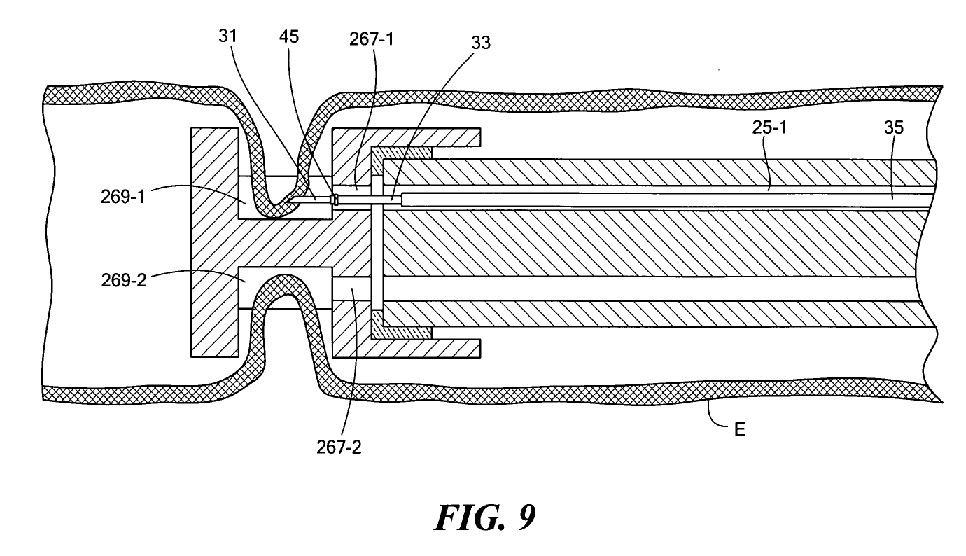

Assembly 251 may be used in the manner illustrated in FIG. 9. As can be seen, the distal end of assembly 251 may be positioned generally centrally within the esophagus E or other tissue of interest of the patient. As suction is applied, fluid (e.g., air) may be drawn proximally through openings 267-1 and 267-2 and then through channel 25-1, thereby causing tissue to be drawn radially inwardly, in substantially a 360° manner, and stabilized against surfaces 269-1 and 269-2. Needle 31 may then be extended distally for insertion into the tissue that has been drawn towards surface 269-1. As can be seen, assembly 251 may permit one to control the depth of needle penetration.

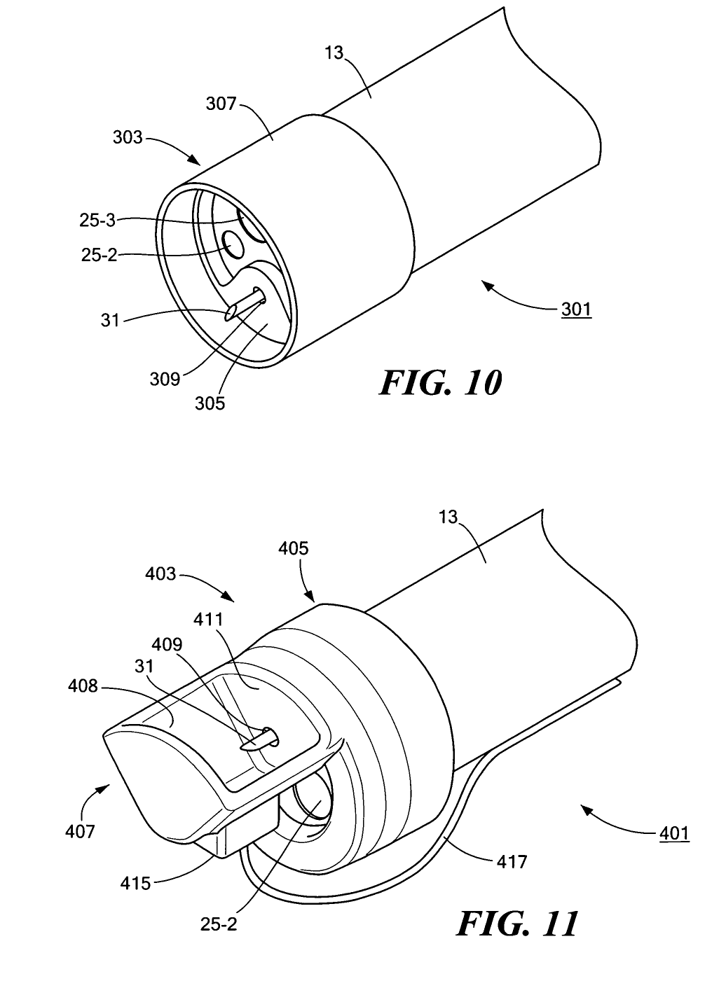

Referring now to FIG. 10, there is shown a fragmentary perspective view of a sixth embodiment of an endoscopic injection needle assembly constructed according to the teachings of the present invention, said endoscopic injection needle assembly being represented generally by reference numeral 301.

Assembly 301 may be similar in most respects to assembly 11, the principal difference between the two assemblies being that, whereas assembly 11 may include an endoscopic hood 17, assembly 301 may include an endoscopic hood 303. Endoscopic hood 303 may be generally similar to hood 17, the two hoods differing primarily in that hood 303 may include a tab 305 extending radially inwardly a short distance from the side wall 307 of hood 303 to cover the working channel of endoscope 13. An opening 309 may be provided in tab 305, opening 309 being appropriately dimensioned to permit needle 31 to be selectively extended therethrough while, at the same time, permitting fluid to be drawn proximally around the outside of needle 31 and into the working channel of endoscope 13.

Assembly 301 may be used in a fashion similar to that discussed above for assembly 11. Like assembly 1, assembly 301 may provide one with the ability to stabilize the targeted tissue and to control the depth of needle penetration.

Referring now to FIG. 11, there is shown a fragmentary perspective view of a seventh embodiment of an endoscopic injection needle assembly constructed according to the teachings of the present invention, said endoscopic injection needle assembly being represented generally by reference numeral 401.

Assembly 401 may be similar in many respects to assembly 11. One difference between the two assemblies may be that, whereas assembly 11 may include an endoscopic hood 17, assembly 401 may include an endoscopic hood 403. Hood 403 may be a unitary member shaped to include a proximal portion 405 and a distal portion 407. Proximal portion 405, which may be generally tubular in shape, may be fixedly mounted, preferably by a friction-fit or by a gasket or O-ring, onto distal end 23 of endoscope 13. Distal portion 407, which may extend distally from only a portion of proximal portion 405 so as not to obstruct viewing channel 25-3, may be a generally trough-shaped structure having a side cavity 408. An opening 409 may be formed in a wall 411 separating proximal portion 405 and distal portion 407, opening 409 being aligned with the working channel of endoscope 13 and being adapted to permit needle 31 to be selectively extended therethrough while, at the same time, permitting fluid to be drawn proximally around needle 31 and into the working channel of endoscope 13.

Assembly 401 may further include an ultrasound probe 415 for use in imaging the tissue drawn into distal portion 407 through cavity 408 and for monitoring the insertion of needle 31 into said tissue. Probe 415 may be mounted on distal portion 407 of hood 403 opposite cavity 408. A cable 417 may be coupled to probe 415, the proximal end of cable 417 being drawn through the working channel of the endoscope or, as shown, carried along the outside of endoscope 13. As can be appreciated, hood 403 may be made of material compatible for use with probe 415.

Assembly 401 may be used in a fashion similar to that discussed above in connection with assembly 151, with distal end 407 of hood 403 being oriented relative to the esophagus so that side cavity 408 may be placed flush against the esophageal wall, thereby permitting tissue to be drawn therethrough. Like assembly 151, assembly 401 may provide one with the ability to stabilize the targeted tissue and to control the depth of needle penetration.

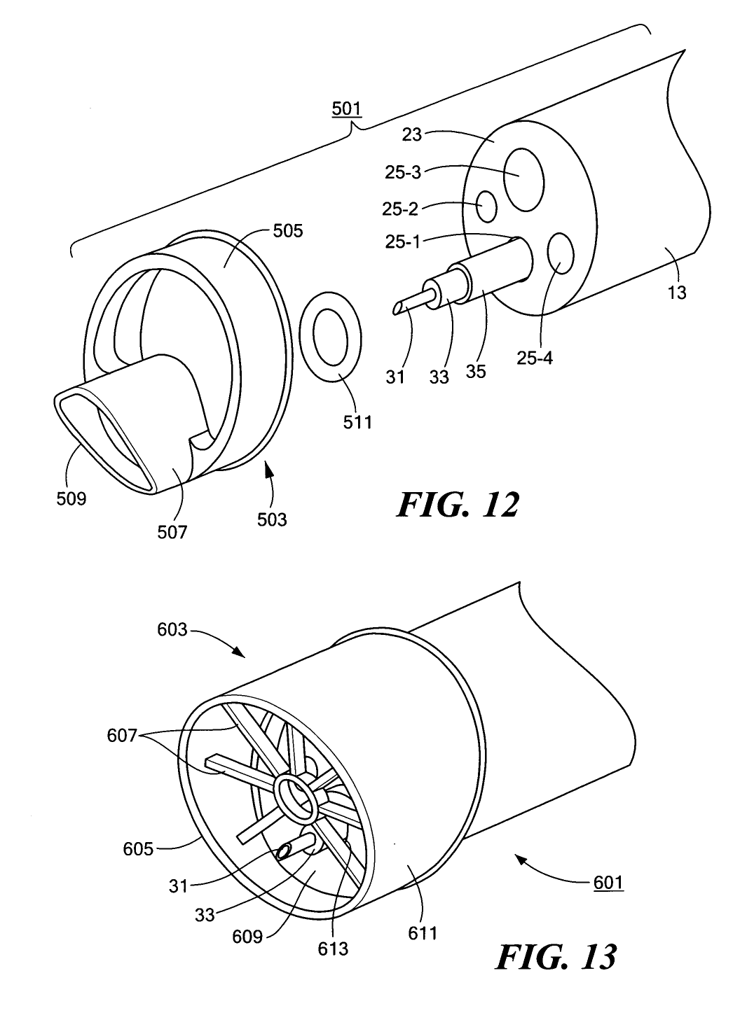

Referring now to FIG. 12, there is shown a fragmentary exploded perspective view of an eighth embodiment of an endoscopic injection needle assembly constructed according to the teachings of the present invention, said endoscopic injection needle assembly being represented generally by reference numeral 501.

Assembly 501 may be similar in many respects to assembly 11. One difference between the two assemblies may be that, whereas assembly 11 may include an endoscopic hood 17, assembly 501 may include an endoscopic hood 503. Hood 503 may be a unitary member shaped to include a proximal portion 505 and a distal portion 507. Proximal portion 505, which may be generally tubular in shape, may be fixedly mounted over distal end 23 of endoscope 13. Distal portion 507, which may extend distally from only a portion of proximal portion 505 so as not to obstruct channels 25-2, 25-3 or 25-4, may be a generally tubular member having a beveled distal end 509. A sealing ring 511 may be positioned between hood 503 and endoscope 13 so as to form a tight seal around working channel 25-1.

Assembly 501 may be used in a fashion similar to that discussed above for assembly 11. Like assembly 1, assembly 501 may provide one with the ability to stabilize the targeted tissue and to control the depth of needle penetration.

Referring now to FIG. 13, there is shown a fragmentary perspective view of a ninth embodiment of an endoscopic injection needle assembly constructed according to the teachings of the present invention, said endoscopic injection needle assembly being represented generally by reference numeral 601.

Assembly 601 may be similar in many respects to assembly 101, the principal difference between the two assemblies being that, whereas assembly 101 may include an endoscopic hood 103, assembly 601 may include an endoscopic hood 603. Hood 603 may be similar to hood 103 in that hood 603 may have a beveled distal end 605. Hood 603 differs principally from hood 103 in that hood 603 may additionally include a plurality of slats 607 provided in the interior of hood 603 proximate to distal end 605. Slats 607 may be arranged in a web-like pattern to limit the volume of tissue that may enter hood 603. (It should be appreciated that, instead of a web-like pattern, the slats could be arranged in a grid or in any other pattern that similarly functions to limit the depth to which tissue may enter.) In addition, hood 603 may include, positioned proximally relative to slats 607, a tab 609 extending radially inwardly a short distance from a side wall 611 of hood 603 to cover the working channel of endoscope 13. An opening 613 may be provided in tab 609, opening 613 being appropriately dimensioned to permit needle 31 and inner catheter 33 to be selectively extended therethrough while, at the same time, permitting fluid to be drawn proximally around the outside of inner catheter 33 and into the working channel of endoscope 13.

Assembly 601 may be used in a fashion similar to that discussed above for assembly 101. Like assembly 101, assembly 601 may provide one with the ability to stabilize the targeted tissue and to control the depth of needle penetration.

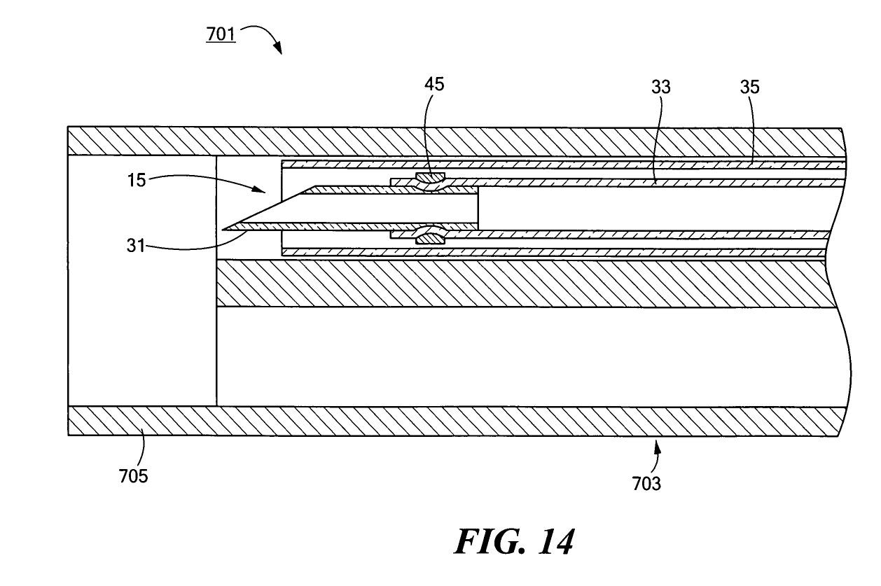

Referring now to FIG. 14, there is shown a fragmentary longitudinal section view of a tenth embodiment of an endoscopic injection needle assembly constructed according to the teachings of the present invention, said endoscopic injection needle assembly being represented generally by reference numeral 701.

Assembly 701 may be similar in many respects to assembly 11, the principal difference between the two assemblies being that, whereas assembly 11 may include endoscope 13 and endoscopic hood 17, assembly 701 may include an endoscope 703 that includes an integrally formed hood portion 705.

Assembly 701 may be used in a fashion similar to that discussed above in connection with assembly 11. Like assembly 11, assembly 701 may provide one with the ability to stabilize the targeted tissue and to control the depth of needle penetration.

It should be noted that, although the endoscopic injection needle assemblies of the present invention have been described above as being used to inject implant material into LES tissue, such assemblies are not limited to injecting implant materials nor are they limited to injecting materials into LES tissue. In fact, the assemblies of the present invention may be used to inject drugs or other agents into any area of the body including, but not limited to, the GI tract, such as the pyloric sphincter, stomach wall, small and large intestinal walls, colon wall, biliary duct, pancreatic duct, etc.

In addition, it should be noted that any of the assemblies described herein may include an ultrasound probe, a laser fiber, an RF or other ablation probe or other endoscopic devices including, but not limited to, snares, biopsy forceps, hemostasis probes, and ligation bands.

Moreover, it should be noted that with assemblies that involve side suction, the needle may bend sideways to pierce the tissue and may be curved as well.

The embodiments of the present invention described above are intended to be merely exemplary and those skilled in the art shall be able to make numerous variations and modifications to it without departing from the spirit of the present invention. All such variations and modifications are intended to be within the scope of the present invention as defined in the appended claims.

What is claimed is:

1. An endoscopic injection needle assembly comprising:
(a) an endoscope, the endoscope including a working channel, the working channel having a proximal end and a distal end;
(b) an injection needle, at least a portion of the injection needle being disposed within the working channel, the injection needle being sized relative to the working channel to permit fluid to flow around the injection needle in the working channel;
(c) a hood, the hood having a proximal end and a distal end, the hood extending distally from the distal end of the endoscope, the hood being in fluid communication with the working channel and having an opening through which fluid may be drawn into the hood; and
(d) a suction source, the suction source being applied to the proximal end of the working channel to cause fluid to be drawn into the hood through the opening and then proximally through the working channel.

2. The endoscopic injection needle assembly as claimed in claim 1, wherein the opening is provided in the distal end of the hood.

3. The endoscopic injection needle assembly as claimed in claim 2, wherein the hood has a straight distal end.

4. The endoscopic injection needle assembly as claimed in claim 2, wherein the hood has a beveled distal end.

5. The endoscopic injection needle assembly as claimed in claim 1, wherein the opening is a side window provided at an intermediate point between the proximal end and the distal end of the hood.

6. The endoscopic injection needle assembly as claimed in claim 5, wherein the hood has a closed distal end.

7. The endoscopic injection needle assembly as claimed in claim 1, wherein the injection needle comprises a hollow needle, an inner catheter and an outer catheter, the hollow needle being fixedly mounted within a distal end of the inner catheter, the hollow needle and the inner catheter being slidably mounted within the outer catheter.

8. The endoscopic injection needle assembly as claimed in claim 1, wherein the injection needle comprises a hollow needle and a catheter, the hollow needle being fixedly mounted within the hood, the catheter being coupled to the hollow needle.

9. The endoscopic injection needle assembly as claimed in claim 8, wherein the hood includes a side wall and at least one slat extending inwardly from the side wall, the hollow needle being mounted on the at least one slat.

10. The endoscopic injection needle assembly as claimed in claim 9, wherein the hood has a beveled distal end.

11. The endoscopic injection needle assembly as claimed in claim 1, wherein the hood has a proximal portion, an intermediate portion, and a distal portion, the proximal portion comprising a side wall and a transverse wall, the opening being provided in the transverse wall, the proximal portion being mounted on the endoscope with the transverse wall spaced from the distal end of the working channel, the intermediate portion having a reduced outer diameter as compared to the proximal portion.

12. The endoscopic injection needle assembly as claimed in claim 11, wherein the distal end of the hood is solid.

13. The endoscopic injection needle assembly as claimed in claim 1 further comprising an ultrasound probe, the ultrasound probe being mounted on the hood.

14. The endoscopic injection needle assembly as claimed in claim 1, wherein the hood comprises a proximal portion and a distal portion, the distal portion having a reduced outer diameter as compared to the proximal portion and being shaped to include a trough, the opening being provided in a wall separating the proximal portion and the distal portion, the opening being aligned with the working channel and being adapted for a needle of the injection needle to be inserted therethrough.

15. The endoscopic injection needle assembly as claimed in claim 1, wherein the hood includes a tubular proximal portion and a tubular distal portion, the tubular distal portion having a reduced outer diameter as compared to the tubular proximal portion.

16. The endoscopic injection needle assembly as claimed in claim 15, wherein the tubular distal portion has a beveled distal end.

17. The endoscopic injection needle assembly as claimed in claim 15, wherein the endoscope further includes a viewing channel and wherein the tubular proximal portion has a viewing opening aligned with the viewing channel.

18. The endoscopic injection needle assembly as claimed in claim 1, wherein the hood includes a side wall and a plurality of slats extending inwardly from said side wall.

19. The endoscopic injection needle assembly as claimed in claim 18, wherein the hood has a beveled distal end.

20. A method of making an injection, the method comprising the steps of:
(a) providing an endoscope assembly, the endoscope assembly including an endoscope and a hood, the endoscope including a proximal end, a distal end and a working channel extending from the proximal end of the endoscope to the distal end of the endoscope, the hood being mounted on the endoscope and extending distally beyond the distal end of the endoscope, the hood having a proximal end, a distal end, and an opening through which fluid may be drawn into the hood;
(b) inserting the hood and the distal end of the endoscope into a patient;
(c) positioning the hood in the area of a targeted tissue;
(d) providing an injection needle, the injection needle having a proximal end and a distal end and including a hollow needle and a catheter, the catheter being fluidly coupled to the hollow needle, the distal end of the injection needle being positioned within the working channel of the endoscope so that the hollow needle is in the vicinity of the distal end of the endoscope, the injection needle being sized relative to the working channel to permit fluid to flow around the injection needle in the working channel;
(e) applying a vacuum to a proximal end of the working channel to draw fluid into the hood through the opening and then proximally through the working channel of the endoscope, thereby causing tissue to be drawn into the hood;
(f) inserting the hollow needle of the injection needle into the tissue drawn into the hood; and
(g) dispensing a desired substance into the inserted tissue through the catheter and the hollow needle.

21. The method as claimed in claim 20, wherein the opening in the hood is provided in the distal end of the hood.

22. The method as claimed in claim 21, wherein the hood has a straight distal end.

23. The method as claimed in claim 21, wherein the hood has a beveled distal end.

24. The method as claimed in claim 20, wherein the opening is a side window provided at an intermediate point between the proximal end and the distal end of the hood.

25. The method as claimed in claim 24, wherein the hood has a closed distal end.

26. The method as claimed in claim 20, wherein the hood positioning step comprises contacting the opening of the hood with tissue surrounding the targeted tissue.
